(19) United States
(12) Patent Application Publication
(10) Pub. No.: US 2007/0166186 A1
(43) Pub. Date: Jul. 19, 2007

(54) Descenting apparatus and method

(76) Inventor: Michael J. Stec, Youngsville, PA (US)

Correspondence Address: JAMES C. SIMMONS, 11 FALMOUTH LANE, WILLIAMSVILLE, NY, 14221, US

(21) Appl. No.: 10/589,057

(22) PCT Filed: Feb. 11, 2005
(86) PCT No.: PCT/US05/04322

§ 371 Date: Aug. 11, 2006

Related U.S. Application Data
(60) Provisional application No. 60/543,505, filed on Feb. 11, 2004.

(51) Int. Cl. A61L 9/04; A61L 9/12

(52) U.S. Cl. 422/5; 422/292; 422/305; 422/186.12

(57) ABSTRACT

Descenting apparatus and method. An ozone generator (32) is disposed within and mounted to a portable denclosure (10) for releasing ozone within said enclosure for descenting a hunter's clothes (12) and hunting gear (16) or for descenting a dog training cloth to which a scent is thereafter applied. In a hunting lodge, a stand-along room (50) has an opening (54) to the outside of the lodge and is sealed against entrance of odors from the remainder of the lodge and environmentally controllable with air only from outside the lodge. An ozone generator (52) is mounted to a wall (78) thereof for releasing ozone for descenting a hunter and the hunter's clothes and hunting gear (74).

The present invention relates generally to descenting (removal of scents) apparatus and methods, examples of which are found in U.S. Pat. Nos. 4,309,388; 4,867,052; 4,941,270; 5,087,426; 5,433,919; 5,468,454; 5,484,472; 5,514,345; 5,547,476; 5,667,564; 5,681,355; 5,762,648; 5,766,560; 5,789,368; 5,911,957; 5,931,014; 6,007,770; 6,134,806; 6,149,038; 6,156,268; 6,163,098; 6,284,204; 6,312,507; 6,355,216; 6,379,435; 6,503,547; 6,564,591; 6,565,805; and 6,576,190, and published U.S. patent application 2003/0044308, all of which are incorporated herein by reference.

More particularly, the present invention relates to descenting apparatus and methods for use by hunters and dog trainers.

Deer and other wild game have very keen senses of smell, which are used for detecting the presence of a human or other danger as well as for locating suitable companions. If a hunter allows a deer to prematurely detect his presence, he has lost the prey. It is not just one's own smells that a hunter needs to worry about. For example, a deer on a hill-top may be tipped off that something is wrong (danger) by swamp or vegetative scents picked up in a previous day's hunting or by scents picked up from a cabin or lodge in which the hunter is staying.

Many efforts have been made to remove scents on or about a hunter so as to reduce the possibility of detection by wild game that is being hunted. Such efforts are discussed in Hunting Scent Book by Wildlife Research Center, Inc. of Anoka, Minn, 1991. As discussed therein, the use of scents or the masking or elimination of scents can greatly increase a hunter's chances of success, and scent eliminating soaps and sprays are suggested. Activated charcoal has also been provided in hunters' clothing for the purpose of eliminating odors. See, for example, U.S. Pat. Nos. 5,539,930; 5,790,987; 6,009,559; and 6,134,718, all of which are incorporated herein by reference. These methods for descenting hunters are cumbersome, difficult to use, not thorough enough, or otherwise inadequate.

In order to train dogs for drug detection or to trace different scents, it is considered important that the scent for which the dog is being tested be put on descented cloth to provide more reliable test results, i.e., you can then prove exactly what the dog has accomplished.

It is accordingly an object of the present invention to thoroughly and easily descent the clothes and other personal effects of hunters.

It is another object of the present invention to achieve reliable test results when training dogs for finding or tracing different scents.

In order to thoroughly and easily descent the clothes and other personal effects of hunters, in accordance with the present invention, the hunter's clothes and other personal effects to be worn or carried on a hunting trip are placed in a portable enclosure wherein they are descented by ozone released by an ozone generator within the enclosure.

In order to prevent a hunter from leaving on a hunting trip contaminated with scents from the lodge in which he is staying, in accordance with the present invention, a descenting room is provided in the lodge wherein the hunter's clothing and effects are subjected to ozone for descenting, the descenting room having an outside door which allows the hunter to go directly outdoors for hunting after being descented without having to go into other rooms or areas of the lodge.

In order to achieve reliable test results when training dogs for finding or tracing different scents, in accordance with the present invention, cloths used for training are descented by placement in an enclosure wherein they are descented by ozone released by an ozone generator within the enclosure.

The above and other objects, features, and advantages of the present invention may be found in the following detailed description of the preferred embodiments thereof when read in conjunction with the accompanying drawings wherein the same reference numerals denote the same or similar parts throughout the several views.

### BRIEF DESCRIPTION OF THE DRAWINGS

FIG. 1 is a perspective view with a portion broken away of a portable enclosure which embodies the present invention.

FIG. 2 is a schematic plan view of a lodge which embodies the present invention.

FIG. 3 is a perspective view of a descenting room in the lodge.

FIG. 4 is a schematic view of an enclosure, which embodies the present invention, for the training of dogs.

### DETAILED DESCRIPTION OF THE PREFERRED EMBODIMENTS

Referring to FIG. 1, there is illustrated generally at 10 a portable closet (enclosure) which may be carried by a hunter on a hunting trip and placed in a lodge or cabin in which he is staying. The closet or other enclosure is sized to be portable for carrying in a personal vehicle such as a hunter's pick-up truck (as opposed to a larger commercial vehicle) without compacting thereof into a compact shape so that the hunter's clothes and hunting gear are receivable therein during the porting thereof. For the purposes of this specification and the claims, a “hunter” is defined as including hunters of other people such as in a war-time situation as well as hunters of wild game and other animals and is meant to also include photographers, and “hunt” or “hunting” is defined as including the hunting or searching for other people such as in a war-time situation as well as wild game and other animals for the purposes of photographing or capturing or killing or observing them.

The closet 10 conventionally has a steel frame with a clear PVC cover providing 4 walls, or is otherwise suitably constructed and/or with different materials. The particulars of construction of the closet 10 are not further described herein since they form no part of the present invention and are well known to those of ordinary skill in the art to which this invention pertains. The closet 10 should be large enough to hang the hunter's hunting clothes 12 and to contain on the floor 20 his hunting gear, such as a gun 14 and boots 16, and any other personal effects thereby desirably allowing space for descenting not only the hunter's clothes but also his effects which he will carry when hunting. For example, the closet 10 may have a height of about 64 inches, a width of about 35.5 inches, and a depth of about 19.5 inches. A rod 22 is suitably fixed to the steel frame to extend horizontally across the closet 10 a short distance below the ceiling 24 for receiving the hangers 26 for the clothes 12, as is conventional for closets. A zipper 28 provides an opening to the closet 10. For increased portability, the closet 10 is desirably provided with wheels 30. An example of a suitable closet is a 36-inch Whitmor rolling clothes closet marketed by Earle Industries, Inc. of Earle, Ariz.

In order that the closet 10 may be used for descenting the clothes 12 and effects 14 and 16 in preparation for hunting, an ozone generator 32, preferably compact, is suitably placed within the closet 10, preferably near the top of the closet 10 for better distribution of ozone within the closet 10. Ozone is responsible for the clean fresh smell after a thunderstorm. Ozone (a molecule of 3 atoms of oxygen instead of the usual 2 atoms making up a molecule of oxygen) combines with (oxidizes) scent-producing molecules, cleanly and effectively neutralizing them while the ozone is cleanly reduced to a molecule of oxygen, leaving no residue. As a result, the scent is not merely covered up or masked but is effectively eliminated. In order to provide effective descenting treatment for the above-sized closet without unduly raising living area ozone level, the ozone generator 32 desirably has a variable ozone output of about 30 to 70 mg per hour, and is desirably of light weight and compact for use in the portable closet 10. The ozone generator 32 desirably also has ionization means (commonly known in the art). Desirably, the ozone generator 32 is of a type which has low-maintenance corona discharge plates (which are commonly known in the art) through which oxygen passes for producing ozone, the plates advantageously not requiring periodic scrubbing (as may be required by other types of discharge plates which may be tarnished by the electric charge passing between them).

An example of a suitable ozone generator is the Bora Living Air electronic air-purification system, a product of EcoQuest international (formerly Alpine Industries) of Greenville, Tenn., which has a specified ozone output of 70 mg per hour and desirably has small weight (3 pounds) and size (7.25×5.25×2 inches) and has needle-point ionization. Other examples of suitable ozone generators include models XL-15S and XL-15S Plus (which have radio-wave as well as needle-point ionization) and the Peak III (which has needle-point ionization) systems also of EcoQuest international. Additional examples are the Biozone TravelAire 250 air purifier (which has photoionization) marketed by Biozone Scientific, Inc. of Vero Beach, Fla., and the Edenair DC-12 odor removal model ozone generator marketed by Jenesco Inc. of Amherst, N.H.

Preferably, the ozone generator 32 electrical supply is adapted for D.C. (direct current) as well as A.C. (alternating current) operation; for example, it may be provided to be plugged into a D.C. cigarette lighter adapter, as provided by the above Peak III system.

The ozone generator 32 is attached to the inside of the closet 10 preferably by a clip 34 on the upper wall of the ozone generator mounting the ozone generator 32 on one end of the bar 22 so that it is permanently but detachably mounted to be “out of the way” of the hanging of clothes. A vertical conduit or channel, illustrated at 36, is preferably formed in or to the nearest closet wall 18 for receiving the electric cord 38 for the ozone generator 32, leading it to the bottom of the closet 10 and exteriorly thereof so that it can be plugged into a wall outlet. The bottom of the ozone generator 32 is preferably suitably clipped to the conduit 36 as by clip 40 so that the ozone generator 32 does not move around within the closet during porting of the closet 10. It should be understood that the ozone generator 32 may be otherwise suitably mounted within the closet 10.

The enclosure of the present invention may alternatively be a container or box such as, for example, a suitcase or a Tupperware container or other box which is receivable on the back of a pick-up truck. The Tupperware container may, for example, have a 25 to 45 gallon capacity. For example, the Tupperware container may have a size of 24×16×19 inches. As with the closet 10, the ozone generator should preferably be mounted near the top of the enclosure for good dispersal, with, for example, a bracket or in a bracketed cage. In addition to its use by hunters, the enclosure of the present invention may be used by business or other travelers or at home.

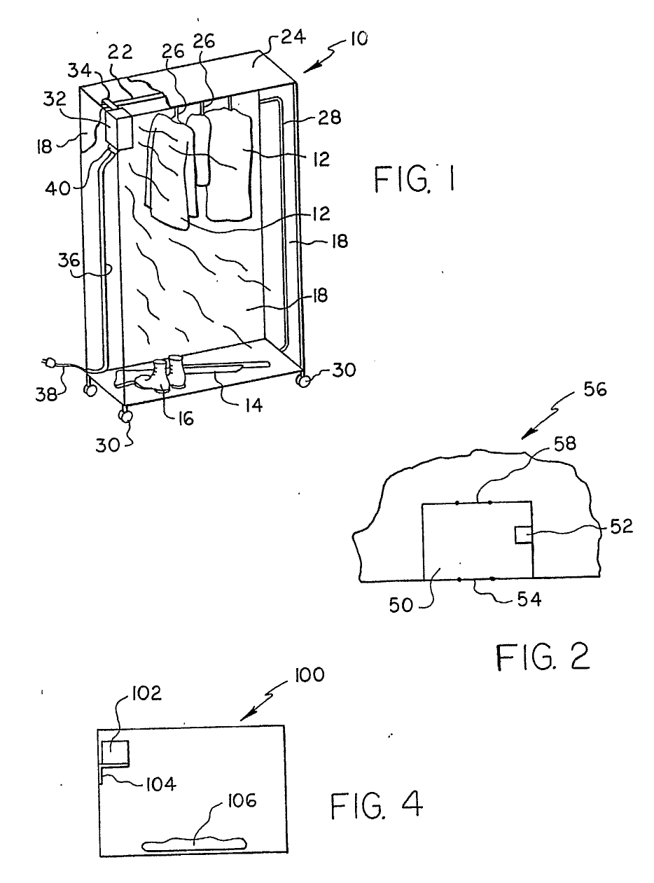

Even after a hunter's clothes and effects have been descented, he may still get odors on himself from the lodge in which he is staying as he leaves to go hunting. Referring to FIG. 2, in order to prevent this from occurring, in accordance with the present invention, a stand-alone room, illustrated at 50, with a variable level ozone ionization generator 52 and an outlet door 54 directly to the outside of the lodge, illustrated generally at 56, is provided. For ease of illustration of the articles therein, the walls, floor, and ceiling of the room 50 are shown in phantom lines. An interior door 58 provides entrance into or from the remainder of the lodge 56, the door 58 being suitably sealed to prevent entrance into the room 50 of odors from the remainder of the lodge. The ozone generator 52, which has a capacity suitable for the size of the room 50 and is sized accordingly, is otherwise similar to the ozone generator 32. The ozone generator 52 is desirably permanently mounted to a wall near the upper part of the room 50 for good dispersal, as illustrated at 78, of ozone within the room 50. The ozone generator air intake (of air from within the room 50) and dispersal fan are illustrated at 80 and 82 respectively.

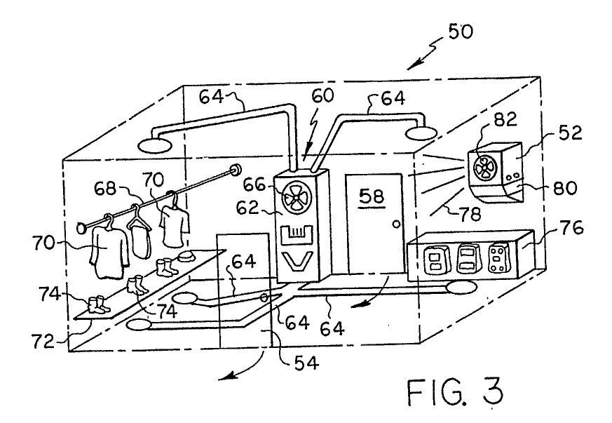

Referring to FIG. 3, in order to dry any wet clothes of the hunter as well as to maintain a comfortable environment within the room 50 so that it is a stand-alone room without dependence on air from the remainder of the lodge 56, the room 50 is desirably environmentally controlled with heat, humidity, and air circulation equipment, illustrated generally at 60, including a heater 62 with humidity control, air circulation conduits 64, and a fan 66, all controlled with programmable devices to regulate air using outdoor air if needed to create positive air flow, and all being conventional and therefore not described in greater detail herein. The room 50 also contains a clothes rack 68 for the hunter's clothes 70, a rack 72 for the hunter's boots/shoes 74, and a gear storage enclosure 76. Thus, the stand-alone ozone treated room 50 allows the hunter and his clothes and hunting gear and other effects to be descented so that he can leave the room 50 through outside door 54 directly to the outside and his truck for hunting without the possibility of his becoming contaminated with odors from the remainder of the lodge 56 which may otherwise happen if he had to pass therethrough to go hunting.

The provision of a stand-alone room 50 at a hunting lodge, in addition to being more effective at keeping the hunter odor-free for hunting, also advantageously eliminates the need of the hunter to carry a portable ozone-treatment enclosure along with him.

The hunter may also apply a suitable scent or scent mask to his body to prevent detection of his scent from sweating and the like. After his clothes and effects have been descented, a hunter may apply a selected desired scent such as, for example, that of a female deer to attract wild game.

During testing on search and rescue dogs, the dogs uniformly did not appear to be able to pick up human scent from human scented-cloth that had been treated with ozone to descent it, thus confirming that treatment of human scented clothing with ozone does indeed eliminate human scent from clothing.

In the training of search and rescue dogs as well as military dogs, it is considered to be important to put a target scent such as a drug or human scent on a descented or scent-free cloth in order to obtain more reliable or truer results, i.e., a test using a scent on a descented cloth will more reliably prove exactly what the dog has accomplished. Referring to FIG. 4, in order to provide descented cloths for the training of search and rescue and military dogs as well as other animals, in accordance with the present invention, a decontamination or descenting chamber or enclosure, illustrated generally at 100, is provided which has an ozone generator 102 permanently affixed, detachably, to a wall on the inside thereof (preferably near the top to aid in ozone dispersal within the chamber 100) such as by a bracket 104. The ozone generator 102 may be similar to ozone generator 32. Like the ozone generator 32, a suitable conduit may be provided as necessary to receive the electrical cord therefor, which is led to a source of power outside the chamber.

In order to train a search and rescue or military dog, in accordance with the present invention, a training cloth 106 is first descented by placement in chamber 100, the ozone generator 102 is then operated to thereby descent the cloth 106 by subjecting it to ozone, then a test scent is applied to the descented cloth 106, and the dog is tested/trained with the test scent on the previously descented cloth 106. For the purposes of this specification and the claims, the training of dogs or other animals is meant to include the testing thereof.

It should be understood that, while the present invention has been described in detail herein, the invention can be embodied otherwise without departing from the principles thereof, and such other embodiments are meant to come within the scope of the present invention as defined by the appended claims.

1. Portable descenting apparatus comprising an enclosure having a closable opening for receiving items for descenting and sized to be portable for carrying in a personal vehicle, and an ozone generator disposed within and mounted to said enclosure for releasing ozone within said enclosure for descenting the items.

2. Apparatus according to claim 1 wherein said enclosure is a closet sized for receiving hunters' clothing and gear, said closet having a rod positioned for receiving hangers for hanging of clothing, the apparatus further comprising a clip mounting said ozone generator to said rod.

3. Apparatus according to claim 2 further comprising an electric cord connected to said ozone generator for supplying electric power to said ozone generator from a source outside said closet, said closet having a conduit extending along a wall thereof for leading said electric cord from said ozone generator to a bottom of said closet and exteriorly thereof.

4. Apparatus according to claim 3 further comprising a clip attaching said ozone generator to said conduit.

5. Apparatus according to claim 1 further comprising wheels on said enclosure.

6. Apparatus according to claim 1 wherein said ozone generator is adapted for both A.C. and D. C. electrical power operation.

7. Apparatus according to claim 1 wherein said ozone generator is of a type which has ionization means.

8. Apparatus according to claim 1 wherein said ozone generator is of a type which has corona discharge plates through which oxygen passes for producing ozone.

9. (canceled)

10. A method for use by a hunter for descenting clothing and gear of a hunter while on a hunting trip comprising the steps of:
a. carrying on the hunting trip an enclosure in which is received an ozone generator, wherein the ozone generator is mounted to the enclosures;
b. disposing the clothing and the gear in the enclosure; and
c. operating the ozone generator to generate ozone within the enclosure to descent the clothing and gear.

11. A method for training an animal to detect a scent comprising the steps of:
a. disposing at least one item within an enclosure in which is received an ozone generator;
b. operating the ozone generator to generate ozone within the enclosure to descent the item;
c. applying the scent to the descented item; and
d. using the descented item with the scent applied to train the animal to detect the scent.

12. A method according to claim 11 wherein the ozone generator is mounted to the enclosure.

13. In a hunting lodge, a descenting room which is sealable against entrance to said room of odors from the remainder of the lodge and which comprises a door providing an outlet directly to the outside of the lodge, equipment having intake of air only from outside the lodge for controlling environment within said room, and an ozone generator mounted within said room for releasing ozone for descenting a hunter's clothing and gear.

14. A descenting room according to claim 13 wherein said environment control equipment includes heat, humidity, and air circulation equipment using the outside air to create a positive air flow within the descenting room.

15. A descenting room according to claim 13 wherein said room further comprises a door connecting said room with the remainder of the lodge and being sealable to prevent entrance to said room of odors from the remainder of the lodge.

16. (canceled)

17. A method for use by a hunter at a hunting lodge for descenting the hunter and the hunter's clothing and hunting gear comprising the steps of:
a. entering a room of the lodge and disposing the clothing and hunting gear in the room;
b. sealing the room against entrance to the room of odors from the remainder of the lodge;
c. operating equipment within the room including intaking air as needed only from outside the lodge to control the environment within the room;
d. operating an ozone generator which is mounted within the room to generate ozone within the sealed room to descent the hunter and the hunter's clothing and hunting gear; and
e. leaving from the room through a door which opens directly to the outside of the lodge.

18. (canceled)

19. A method according to claim 17 further comprising applying a selected scent to the descented hunter.

20. Apparatus according to claim 1 wherein said ozone generator is attached to said enclosure so as to be permanently mounted to said enclosure.

21. Apparatus according to claim 1 wherein said ozone generator is characterized to provide an ozone output of about 30 to 70 mg per hour.

22. Apparatus according to claim 1 wherein said enclosure is devoid of means for receiving ozone from a source which is exterior of said enclosure.

23. A descenting room according to claim 13 wherein said ozone generator is attached to said room so as to be permanently mounted to a wall within an upper part of said room.
